(19) United States
(12) Patent Application Publication
(10) Pub. No.: US 2008/0008637 A1
(43) Pub. Date: Jan. 10, 2008

(54) Evaporation device and transport system thereof

(75) Inventors: Ryuji Nishikawa, Hsinshu City (TW); Weh-Hao Wu, Taipei City (TW)

Correspondence Address: VENABLE LLP, P.O. BOX 34385, WASHINGTON, DC, 20043-9998, US

(73) Assignee: TPO Displays Corp., Chu-Nan (TW)

(21) Appl. No.: 11/812,895

(22) Filed: Jun. 22, 2007

(30) Foreign Application Priority Data
Jul. 5, 2006 (TW) 095124460

(51) Int. Cl. A61L 9/00

(52) U.S. Cl. 422/305; 901/50

(57) ABSTRACT

A transport system for an evaporation device comprises a robot holding a substrate and a mask disposed on the robot. When the substrate is transported by the robot, the mask covers the substrate to prevent contamination. The robot has a fork holding the substrate, and the mask is disposed thereon. The mask is disposed under the substrate, specifically between the substrate and the fork.

### BACKGROUND OF THE INVENTION

1. Field of the Invention

The invention relates to a transport system for an evaporation device, and in particular to a transport system protecting substrates from contamination during evaporation.

2. Description of the Related Art

A conventional evaporation device, as shown in FIG. 1, comprises a chamber 10, an emission source 20 and a robot 30.

The chamber 10 is a sealed space and can be evacuated to desired pressure by a vacuum pump.

The emission source 20 is disposed on the bottom of the chamber 10 where evaporation material is disposed. The evaporation material can be organic (small molecule or polymer) or inorganic (metal, ceramic or semiconductor). A heater (not shown) is disposed in the emission source 20. Free molecules or vapor of the evaporation material are emitted from the emission source 20 into the chamber 10 due to generated heat. When a substrate S is disposed in an appropriate position, such as directly above the emission source 20, the evaporation material deposit on the substrate S to form a layer of predetermined thickness, providing the substrate S with desired interface characteristics.

The robot 30 for transporting the substrate S into the chamber 10, as shown in FIGS. 2A and 2B, comprises a base 31, two arms 32 and 33 and a fork 34. The base 31 is fixed. The arms 32 and 33 are slidably joined to the base 31 and extend toward the chamber 10. The fork 34 holds the substrate S. FIG. 2A depicts the robot 30 with arms 33 and 34 withdrawn. FIG. 2B depicts the robot 30 with arms 33 and 34 extended. The fork 34 shown in FIGS. 3A and 3B is U-shaped. A plurality of protrusions,35 is formed on the fork 34, on the surface bearing the substrate S. The substrate S is packed in a frame F.

Referring to FIG. 1 again, when the substrate S is transported by the robot 30, masks M1 and M2 cover the emission source 20 until the substrate S reaches the appropriate position, whereby the substrate S is protected from contamination by the evaporation material, with resultant non-uniform thickness of the layer.

As the emission source 20 is continuously heated to maintain the temperature and emission rate, the evaporation material are continually emitted from the emission source 20. When the substrate S is transported into the chamber 10, masks M1 and M2 cover the emission source 20 until the substrate S reaches a desired position, protecting substrate S from contamination by evaporation material.

Even so, free molecules are able to circumvent masks M1 and M2, depositing on substrate S during transport, passing through the fork 34.

### BRIEF SUMMARY OF INVENTION

A detailed description is given in the following embodiments with reference to the accompanying drawings.

An embodiment of a transport system for an evaporation device of the invention comprises a robot holding the substrate and a mask disposed on the robot. When the substrate is transported by the robot, the mask covers the substrate to prevent contamination.

The mask is disposed under the substrate, specifically between the substrate and the fork. The substrate is disposed on the mask. The mask has a recess in which the substrate is disposed.

### BRIEF DESCRIPTION OF DRAWINGS

The invention can be more fully understood by reading the subsequent detailed description and examples with references made to the accompanying drawings, wherein:

FIG. 1 is a schematic view of a conventional evaporation device;

FIGS. 2A and 2B is a schematic view of the robot of FIG. 1, wherein FIG. 2A depicts arms of the robot withdrawn and FIG. 2B depicts arms of the robot extended;

FIG. 3A is a schematic view of the fork of the robot of FIGS. 2A and 2B;

FIG. 3B is a schematic view showing the fork of FIG. 3A holding a substrate;

FIG. 4 is a schematic view of an evaporation device of the invention;

FIG. 5 is a schematic view showing a mask held on the robot; and

FIG. 6 is a cross section of the mask holding a substrate and a frame.

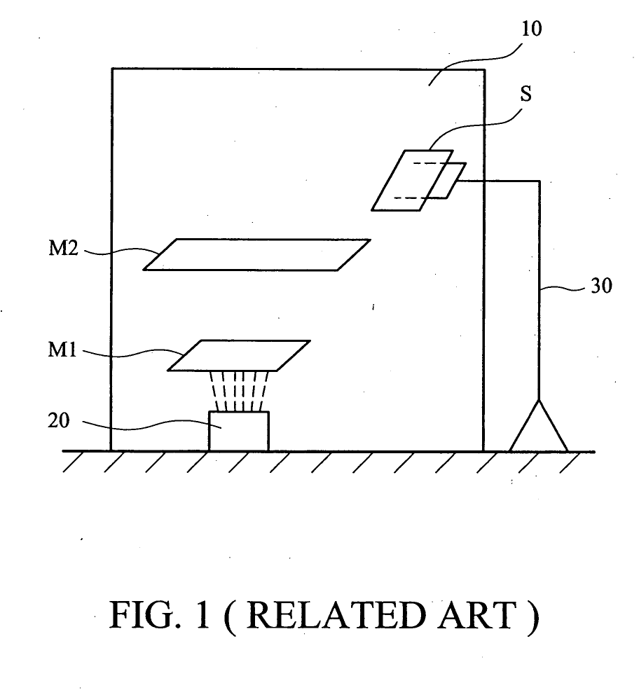

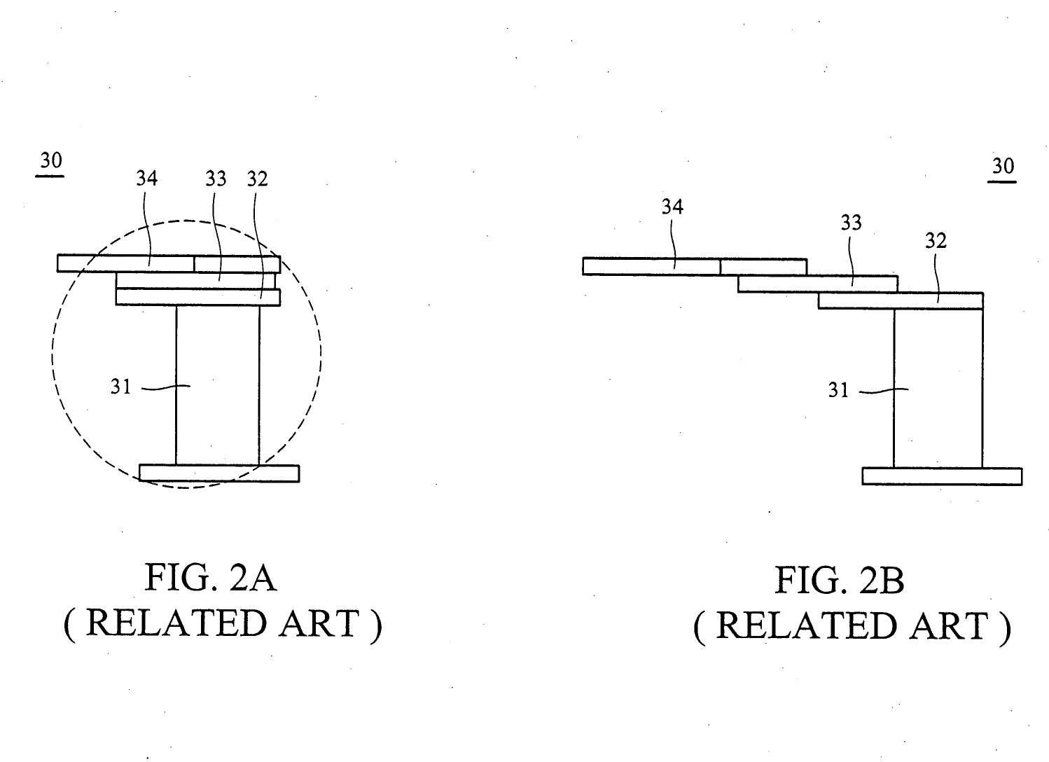

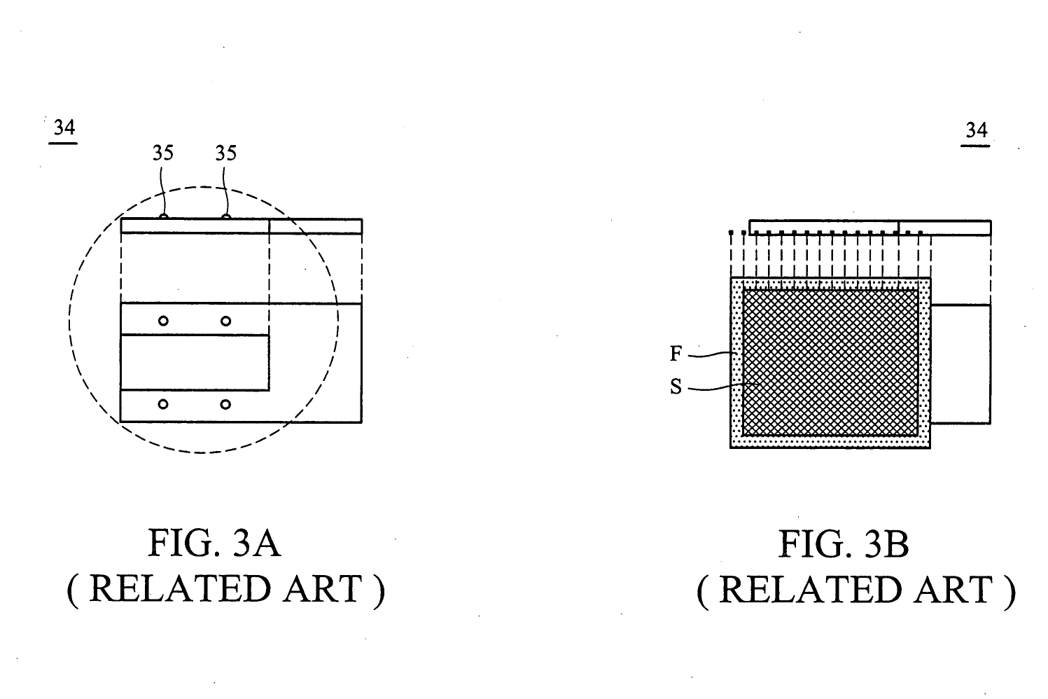

### DETAILED DESCRIPTION OF INVENTION

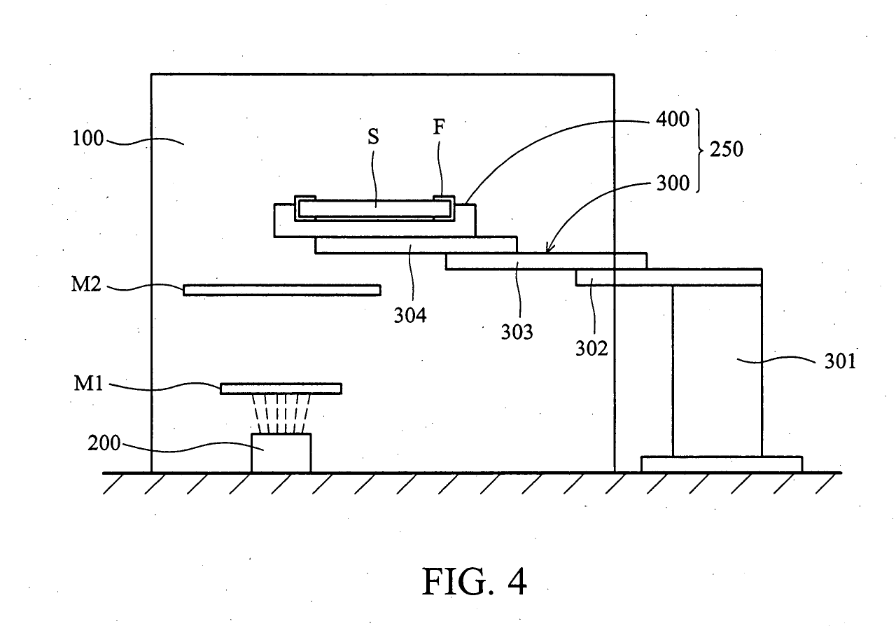

An embodiment of an evaporation of the invention, as shown in FIG. 4, comprises a chamber 100, an emission source 200 and a transport system 250. The transport system 250 comprises a robot 300 and a mask 400.

The chamber 100 is a sealed space, evacuated to desired pressure by a vacuum pump (not shown). The emission source 200 is disposed on the bottom of the chamber 100. Evaporation material is disposed in the emission source 200. The evaporation material can be organic, such as small molecules and polymers, or inorganic, such as metal, ceramic, or semiconductor. A heater (not shown) is disposed in the emission source 200. Free molecules or vapor of the evaporation material are emitted from the emission source 200 into the chamber 100 by generated heat. When a substrate S is disposed in an appropriate position, such as directly above the emission source 200, the evaporation material deposit on the substrate S to form a layer of predetermined thickness, providing desired interface characteristics.

When the substrate S is transported into the chamber 100, masks M1 and, M2 cover the emission source 200 until the substrate S reaches a predetermined position, protecting substrate S from contamination by the evaporation material.

The robot 300 transporting the substrate S into the chamber 100 comprises a base 301, arms 302 and 303 and a fork 304. The base 301 is fixed. The arms 302 and 303 are slidably joined to the base 301, extendable toward the chamber 100. The fork 304 is U-shaped. The robot 300 transports, rolls, or aligns the substrate S. The mask 400 is disposed on the fork 304. The substrate S, accommodated in a frame F, is disposed in the mask 400. The frame F can be omitted.

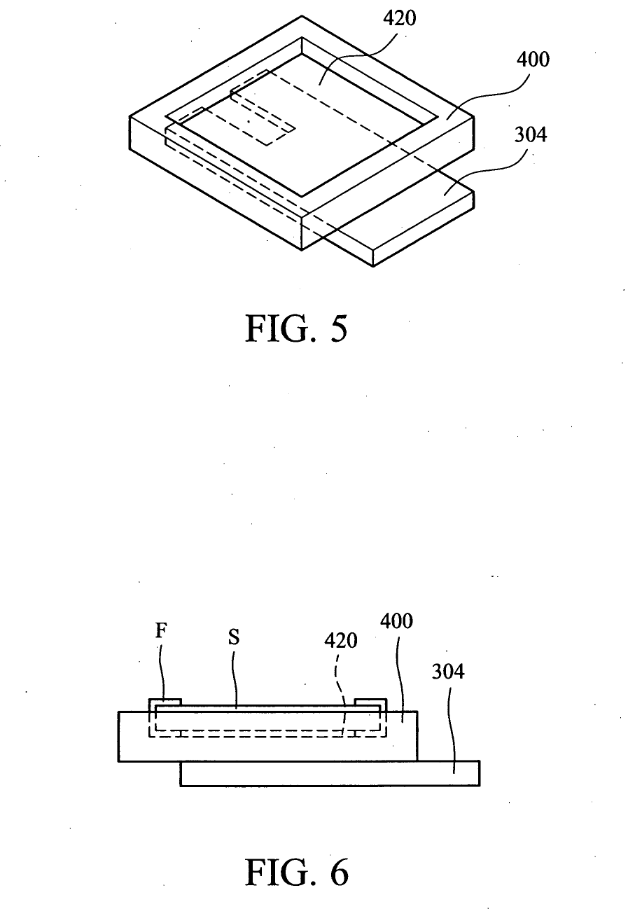

Referring to FIG. 5, the mask 400 is rectangular and has a recess 420. FIG. 6 is a cross section of the mask 400 holding the substrate S and the frame F. The substrate S and the frame F are received in the recess 420. When the substrate S is held by the mask 400 and transported into the chamber 100, the surface for deposition of substrate S is covered by the recess 420, such that evaporation material cannot deposit thereon.

The mask 400 on the robot 300 protects the substrate S from contamination and improper deposition when the substrate S is transported. This enhances thickness uniformity and precision for the deposition layer, and stabilizing interface characteristics.

The transport system 250 can be applied to when a deposition surface of a substrate faces an emission source, such as during manufacture of flat panel displays (FPD), including LCD, OLED, PDP, FED and SED etc.

The transport system of the invention can also be applied to physical vapor deposition (PVD) processes , such as sputtering, thermal evaporation etc., and chemical vapor deposition (CVD) processes, such as PECVD, VUVCVD, MOCVD, ALCVD, LPCVD and thermal chemical deposition etc.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A transport system for transporting a substrate during an evaporation process, comprising:
a robot holding the substrate; and
a mask disposed on the robot, wherein when the substrate is transported by the robot, the mask covers the substrate.

2. The transport system as claimed in claim 1, wherein the robot has a fork holding the substrate, and the mask is disposed on the fork.

3. The transport system as claimed in claim 2, wherein the mask is disposed under the substrate.

4. The transport system as claimed in claim 3, wherein the mask is positioned between the substrate and the fork, and the substrate is disposed on the mask.

5. The transport system as claimed in claim 4, wherein the mask has a recess in which the substrate is received.

6. An evaporation device for a substrate, comprising:
a chamber;
an emission source disposed in the chamber;
a robot holding the substrate into the chamber;
a mask disposed on the robot and positioned between the substrate and the emission source, wherein when the substrate is transported by the robot, the mask covers the substrate.

7. The evaporation device as claimed in claim 6, wherein the robot has a fork holding the substrate, and the mask is disposed on the fork.

8. The evaporation device as claimed in claim 7, wherein the emission source is disposed on the bottom of the chamber, and the mask is under the substrate.

9. The evaporation device as claimed in claim 8, wherein the mask is disposed between the substrate and the fork, and the substrate is disposed in the mask.

10. The evaporation device as claimed in claim 9, wherein the mask has a recess in which the substrate is received.
